(19) United States
(12) Patent Application Publication
(10) Pub. No.: US 2015/0148708 A1
(43) Pub. Date: May 28, 2015

(54) BIOFEEDBACK DURING ASSISTED MOVEMENT REHABILITATION THERAPY

(71) Applicant: Paul Cordo, Portland, OR (US)

(72) Inventor: Paul Cordo, Portland, OR (US)

(73) Assignee: OREGON HEALTH & SCIENCE UNIVERSITY, Portland, OR (US)

(21) Appl. No.: 14/549,731

(22) Filed: Nov. 21, 2014

Related U.S. Application Data
(60) Provisional application No. 61/909,573, filed on Nov. 27, 2013.

(51) Int. Cl. A61B 5/11; A61B 5/00; A61B 5/22

(52) U.S. Cl. CPC A61B 5/1107; A61B 5/224; A61B 5/486; A61B 5/7278; A61B 5/7405; A61B 5/742; A61B 5/7455
USPC 600/587

(57) ABSTRACT

Systems and methods for providing biofeedback during assisted movement rehabilitation therapy while a user is engaged with an assisted movement exercise device are disclosed. In one example approach, a method eliminates passively evoked involuntary torque from the biofeedback. In another example approach, a method eliminates unintentional torque produced by contraction of muscles antagonistic to the assisted movement. In another example approach, a method compensates for the length-tension property of muscle so that the biofeedback relates to the user's level of effort.

### CROSS REFERENCE TO RELATED APPLICATIONS

The present application claims priority to U.S. Provisional Patent Application No. 61/909,573, filed Nov. 27, 2013, entitled “BIOFEEDBACK DURING ASSISTED MOVEMENT REHABILITATION THERAPY,” the entire disclosure of which is hereby incorporated by reference in its entirety.

### ACKNOWLEDGMENT OF GOVERNMENT SUPPORT

This invention was made with United States government support under the terms of grant number AR31017 awarded by the National Institutes of Health. The United States government has certain rights in this invention

### FIELD

The present disclosure relates to the field of rehabilitation of users suffering from motor disorders.

### BACKGROUND

Biofeedback is medically used for many different types of therapeutic interventions, e.g., rehabilitation of victims of stroke and other motor disorders that involve paresis, spasticity, and dyscoordination. For motor disorders such as stroke and spinal cord injury, biofeedback generally provides information to users about some aspects of their movement that they wish to correct or ameliorate in their rehabilitation therapy. One type of such information is the force, or at a joint, the torque, that the user is actively producing with a part of the body, such as a limb. Hereafter and throughout this disclosure, the term “torque” is used herein to refer to either force or torque.

One type of rehabilitation therapy provided for motor disorders is assisted movement, in which an assisted movement device imposes a movement on a joint or joints, and the user is instructed to apply torque in the same direction as the imposed movement. During assisted movement, biofeedback may be provided to users of the assisted movement device in order to inform the users about how well they are complying with the task of assisting the movement, e.g., to provide positive or negative reinforcement of their efforts.

For example, it has been shown that, in individuals who have sustained a neurological injury that severely compromises their ability to move the limbs and, thereby, carry out activities of daily living, treatment of specific impairments such as weakness and spasticity can produce dramatic and sustained improvements in motor control. Such impairments may be treated by a combination of sensory stimulation of the limb and assisted movement. For example, motion may be applied to the treated limb by an electromechanical device while the user attempts to assist volitionally this movement. Concurrent sensory stimulation may be of an electrical or mechanical nature. While the limb is being subjected to this combination of sensory stimulation and applied movement, the user may be provided visual, auditory, or haptic biofeedback of their efforts at assisting the applied movement in order to know when these efforts are successful or not. When users produce torque to assist the movement, the precise manner in which torque biofeedback is presented can influence the effectiveness of the intervention.

However, movement imposed on the body by an assisted movement device, even without the assistance of the user, may produce involuntary torques due to the normal mechanics of the device and to the abnormal mechanics of the user's limb. For example, the user's limb may be spastic or have a contracture, which will resist any movement. However, the load cell or other sensor coupled to the assisted movement device, by virtue of its location in the treatment device, may register not only the user's volitional, or voluntary and intended, torque, but also inertial and frictional torques passively arising from the treatment device itself, as well as from the inertia of the part of the limb moved by the device. The sensor coupled to the assisted movement device may also register any involuntary passive or active torque produced by the user's limb on the device, for example, due to spastic contractions of muscles or contractures of the soft tissues in the limb resulting from long-term immobility. These non-volitional, extraneous torques from the treatment device and the user's limb may lead to feedback to the user of information that does not correspond to the behavior or physiological process that the user is trying to change with clinical intervention. For example, if biofeedback contains extraneous information or noise, this information may make it more difficult for the user to discern how well he/she is responding to the intervention, thereby potentially compromising the effectiveness of the therapy.

Another complicating factor in providing biofeedback of volitional and intentional joint torque, is a tendency for people with a prior stroke or other neuromuscular disorder to contract not only the intended muscle or muscles, but also other unintended muscles at the same time. In fact, these additional, unintended contractions often occur in the very muscle or muscles that directly oppose the intended movement, a phenomenon called “co-contraction.” For example, during an effort to open the hand, the muscles that clench the hand may also be unintentionally contracted, which can prevent the person from opening the hand, or during assisted movement exercise, prevent the biofeedback from showing the user how much torque they are volitionally producing in the hand-opening muscles. If unintentional co-contraction cancels out the user's intentional torque in the opposite direction, the user will not know whether he/she is contracting the correct muscle or muscles, which could compromise the effectiveness of the exercise.

Joint torque generated in the course of assisted movement exercise is categorized in this disclosure into three types. The first is defined herein as “volitional/intentional” torque, meaning that the user is actively generating this torque, and it is in the intended direction. Active torque misdirected to the wrong muscles would still be volitional, but not intentional. The second category of joint torque is defined herein as “passively evoked involuntary” torque, meaning that a device, through movement applied passively to a joint or joints that the user is holding as relaxed as possible, resists the movement as a result of friction, inertia, joint contracture and spasticity. The third is “volitional/unintentional” torque, meaning that the user, through his/her volitional assisted movement, unintentionally causes the muscles opposing the movement to contract, which is termed “co-contraction.” The inventor herein has recognized that biofeedback presented to the user during assisted movement exercise is intended to indicate the user's volitional/intentional torque whereas the other two sources of torque should not be included in driving the user's biofeedback.

Another complicating factor in providing biofeedback of volitional/intentional joint torque during assisted movement of a joint is the so-called “length-tension relationship” of muscle. The length-tension relationship describes a physiological feature of skeletal muscle in which muscle strength changes as function of muscle length. As the muscle shortens during an active movement, the torque produce by that muscle decreases, even with a constant level of activation, due to overlapping chains of myosin protein that interfere with each other. Therefore, for users to maintain a constant level of active joint torque as the muscle shortens in the assisted movement device, they would have to rapidly increase the level of effort and, consequently, the level of muscle activation. Such a requirement could quickly fatigue the user, compromising the effectiveness of the exercise.

### SUMMARY

The present disclosure is directed to systems and methods for providing biofeedback during assisted movement exercise while a user is engaged with an assisted movement exercise device. In particular, biofeedback presented to a user may be manipulated during assisted movement exercise by removing involuntary or unintentional torques from the total torque signal received from the assisted movement exercise device to provide a volitional/intentional torque signal for biofeedback. The volitional/intentional torque signal may then be converted to a signal related more to effort than torque by compensating for the length-tension property of muscle.

For example, a “passive torque” calibration step may be performed to collect calibration measurements from the assisted movement device while the user is passively engaged with the device, i.e., in absence of any volitional torque applied by the user to the device. Following the passive torque calibration step, torque measurements may then be received from the device while the user engages with the device and applies volitional torques to meet target levels during assisted movement exercise. The passive torque calibration data may be used to extract the volitional torque components from the overall torque measurements, e.g. by subtracting passively evoked, non-volitional torque from the overall recorded torque.

In embodiments, any volitional/unintentional joint torque caused by the user co-contracting unintentionally the antagonist muscle or muscles can be removed from the total torque by performing an “unintentional torque” calibration of the recorded torque signal and subtracting the unintentionally produced co-contraction torque from the overall torque. In one example approach, a method for calibrating the unintentional torque for a user comprises increasing the torque bias voltage gradually while the user attempts to move the joint until the recorded torque signal, plus the bias voltage, provides visible biofeedback when the user contracts the correct muscle. Use of the passive torque calibration and unintentional torque calibration procedures ensures that the biofeedback provided to the user indicates only what he/she is doing intentionally. This volitional torque may further be adjusted to compensate for the length-tension property of muscle so that the user is less likely to become fatigued during the assisted movement exercise. This adjusted volitional torque relative to a target level may then be presented to the user, e.g., visually, aurally, and/or haptically, to inform the user about how well he/she is complying with the task of assisting the movement.

In such an approach, the non-volitional components of the torque signal may be removed during assisted movement exercise by, first, measuring some of these non-volitional component in the absence of any assistance from the user, then recalibrating the recorded torque to compensate for passive and co-contraction torques and then, during assisted movement exercise, subtracting these non-volitional torques from the total torque to provide only the intentional component of torque. This intentional component may then be used for the biofeedback. In addition, such approach may compensate for the length-tension property of muscle by increasing the biofeedback gain proportionally to the change in joint angle, for example. This gain change may be set such that the users need only maintain a constant effort at a target level in order to receive positive biofeedback indicating their compliance with the exercise, thereby potentially reducing muscle fatigue during the exercise.

In this way, when an assisted movement treatment device moves a joint or joints, the torque biofeedback presented to the user may be closely related to the user's level of effort, rather than the user's active torque, thereby potentially increasing the effectiveness of the assisted movement exercise. Further such an approach may permit the user to maintain a constant level of effort during joint rotation, rather than having to increase effort as the muscle shortens in order to maintain a constant level of torque. Such an approach may reduce user fatigue and frustration and permit the user to perform the assisted movement exercise for longer periods of time.

This Summary is provided to introduce a selection of concepts in a simplified form that are further described below in the Detailed Description. This Summary is not intended to identify key features or essential features of the disclosed subject matter, nor is it intended to be used to limit the scope of the disclosed subject matter. Furthermore, the disclosed subject matter is not limited to implementations that solve any or all disadvantages noted in any part of this disclosure.

### BRIEF DESCRIPTION OF THE DRAWINGS

FIGS. 1 and 2 schematically show examples of a user engaged with an assisted movement device and receiving biofeedback in accordance with the disclosure.

FIGS. 3 and 4 show an example torque pattern for involuntary and volitional torques, respectively, during ½ cycle of assisted movement exercise and illustrate how volitional and intentional components may be extracted for the user's biofeed back.

FIG. 5 shows an example graph illustrating the length-tension property of muscle and how it may be used to register the user's effort by changing the gain of the biofeedback.

FIG. 6 shows an example method for providing biofeedback during assisted movement exercise while a user is engaged with an assisted movement device in accordance with the disclosure.

FIG. 7 shows a block diagram depicting an embodiment of a computing device.

### DETAILED DESCRIPTION

People who have suffered a loss or limitation of volitional movement due to injury or disease are commonly prescribed and receive rehabilitation therapy. Among many approaches to rehabilitation for volitional movement of the body is “assisted movement” exercise. A rehabilitation user typically performs assisted movement exercise with the help of an electromechanical, or “robotic,” medical device. Such assisted movement exercise devices are typically powered by an electrical motor that produces torque about one or more appendicular joints of the arm, leg, or body axis.

In one embodiment of an assisted movement exercise device, the device provides a constant or time-varying torque in the direction of impoverished joint motion. As the user attempts to apply volitional torque in the desired direction of motion, the device adds torque in the same direction in order to assist the user move the all or part of a limb to a prescribed target location. In this embodiment, the device assists the user with the movement.

In another embodiment of an assisted movement exercise device, the device imposes a prescribed motion on the limb with sufficient torque to ensure that the limb reaches the intended end-point. During the applied motion, the user applies torque to the exercise device in the same direction as it is moving the limb. In this embodiment, the user assists the device with the movement. Both of these embodiments of assisted movement exercise devices typically also employ biofeedback in conjunction with the assisted movement. Biofeedback is designed to inform users of the device about how well they are performing the movement task with the device.

In one embodiment, the biofeedback is visually presented, typically on a display screen, e.g., a computer monitor. In another embodiment of biofeedback, it is aurally presented, typically via loud speakers or headphones. In another embodiment, visual and aural forms of biofeedback are combined. As another example, the biofeedback may be presented via a haptic device that presents tactile feedback to the user. For example, haptic indications may be output via one or more skin stimulators.

Visually and/or aurally and/or haptically presented biofeedback provides the user with either positive reinforcement, when the user performs as instructed, or negative reinforcement, when the user fails to perform the required task at the requested level.

In one embodiment of the biofeedback information, the user is informed about the accuracy of the limb trajectory and/or final position of the limb movement. This embodiment is usually associated with assisted movement exercise in which the device assists the user with the movement.

In another embodiment of the information provided by biofeedback, the user is informed of how much torque they are applying to assist the motion, rather than information about the movement trajectory. This embodiment is usually associated with assisted movement exercise in which the user assists the device, since the exact motion trajectory is prescribed by the device and is unmodified by the user's assistance.

When visually presented biofeedback is used, the information provided by the biofeedback can be presented in more than one format. Characteristics of the form of presentation may include, but are not limited to: the ease of interpretation of the information provided by persons with mild to moderate cognitive limitations, precision of biofeedback equal to the precision required by the user's task, and maintaining the user's interest and attention throughout the period of exercise.

One embodiment of biofeedback format is a simple geometrical graphic that indicates how much torque the user is producing on the exercise device by changing the size and/or position of the graphic in real time on the biofeedback screen. In one example, such a graphic representation may be combined with a target torque level so that the user can be instructed to assist the movement at a level that is challenging, but not so physically taxing that the user becomes too fatigued to continue to the end of an exercise regimen. For example, the target level of torque may be chosen so as to be proportional to the user's current strength and may be adjusted as the user's strength changes over time.

Another embodiment of biofeedback format is a video game. A video game format may reduce predictability of the assisted movement exercise and cause the user to pay closer attention to the task associated with the assisted movement exercise for a longer period of time. The video game format also provides the possibility of increasing the level of difficulty in order to keep the user challenged as his/her physical control over the impaired limb improves. Further, in such an approach, the progress of the user can be measured and updated with a score achieved at the end of each session.

If this type of biofeedback is to be usefully employed on users who are severely paretic, high precision measurements of volitional/intentional torque may be desired. Progress in severely impaired users begins from very low levels of volitional torque, with small increments in strength over time. Without precise measurement of volitional/intentional torque, it may be difficult for a clinician to provide useful biofeedback to the user or to track the user's improvement in strength.

An embodiment of volitional torque biofeedback includes the ability to record, with high precision, the torque impinging on the measurement device, such as a load cell or strain gauge. For example, for severely impaired users, a desired precision of torque measurement may be in an approximate range of 0.2-0.5 Nm at all joints of the limbs. At the same time, the measurement system may also be able to measure larger torques, in order to accommodate users with only moderate or mild limitations of strength and movement. The upper torque limits may further depend on the specific joint and the musculature controlling that joint.

Another embodiment of volitional torque biofeedback distinguishes the volitional component of torque from involuntary or unintentional sources of torque. Involuntary sources include those generated by the assisted movement exercise device, such as frictional and inertial torques, as well as those unintentionally generated by the user, such as exaggerated tone or spasticity, contracture, co-contraction, and inertial torques due to the mass of the limb segment(s) being moved by the assisted movement rehabilitation device.

To remove involuntary components of the total recorded torque, an example method may include, first, recording only the passively evoked, involuntary component, that is, in the absence of any volitional torque. This involuntary component may be measured under the same physical conditions as those encountered during the assisted movement exercise. For example, the involuntary torque and limb kinematics may be both recorded while the device moves the limb with the identical kinematics as used during exercise, and these recorded signals may be used to characterize the relationship between passively evoked involuntary torque and joint angle. Subsequently, during assisted movement exercise, the recorded level of passively evoked involuntary torque may be subtracted, in real-time, from the total recorded torque, with only the volitional component remaining.

A further example method may include calibrating how much oppositional torque is produced when the user volitionally, but unintentionally, co-contracts a muscle or muscles that oppose the intended movement. Subsequently, during assisted movement exercise with biofeedback, the total torque signal recorded by the device can be biased equally and oppositely to the unintended torque resulting from co-contraction. The biofeedback received by the user will then reflect only the torque produced by the user's volitional contraction of the intended muscle or muscles.

In some embodiments, torque biofeedback may be provided to discourage users from overexerting themselves during the exercise. That is, the level of effort required of users may be consistent with not fatiguing their muscles during the exercise session, so that they can complete exercise protocols in the allocated time.

The torque biofeedback presented to the user during assisted movement exercise may include a torque target for each of the user's efforts to assist the applied movement. For example, this torque target may be set to a sufficiently low level, such as 25% of maximum strength, so that the user can continuously perform the exercise for 10-30 minutes or some other selected time duration.

The biofeedback may also present the volitional component of torque adjusted to compensate for the length-tension property of skeletal muscle. The length-tension relationship describes a property of muscle by which, at a constant level of activation, the amount of torque produced by the muscle decreases as the length of the muscle decreases, as occurs when the muscle contracts to move the joint. For a muscle to produce a constant level of torque while moving a joint, the level of muscle activation has to increase at a rate inversely proportional to the length of the muscle. During assisted movement exercise with torque biofeedback, the length-tension property of muscle can lead to muscle and user fatigue.

In order to compensate for the length-tension property of skeletal muscle, the gain of the biofeedback may be controlled such that the gain increases during assisted movement proportionally to the joint angle. For example, as the muscle shortens during assisted movement, the amount of actual torque required for the user to successfully perform the task may be decreased at the same rate as the muscle strength decreases due to muscle shortening. In some examples, successful performance of the task may require only that the user reaches a target level of effort, that is, level of muscle activation, and then maintain this level of effort during the movement.

In some examples, the target level of effort is that required to produce the requisite level of minimized torque when the muscle length is optimal, that is, when it is at its maximum. The muscle(s) the user attempts to contract during assisted movement exercise may be at its longest at the beginning of each movement. Thus, in some examples, the task performed by the user, as reinforced by the biofeedback, is to meet, at the beginning of each assisted movement, a torque target based on the user's current strength, and then to maintain a constant level of effort until the current movement is complete.

In an embodiment of such biofeedback, the biofeedback shows the user, in real time, how much volitional and intentional torque he/she is producing. Torque may be recorded by a load cell or strain gauge coupled to the axis of rotation of the assisted movement exercise device. A torque target, for example, 25% of maximum strength, may be presented to the user along with the torque biofeedback. A target level of 25% of maximum may be challenging to the user, but may not fatigue the user. A superposition of the torque biofeedback and the torque target may be presented to the user to show the user whether he/she is producing torque in the desired direction and whether the current effort level undershoots or reaches the target level. Preferably, in repetitive and cyclical, assisted movement exercise, a different torque target is presented in each direction of movement, since the strength may differ in the muscles on opposite sides of a joint. In some examples, a user's task may include exceeding the target level each time the movement of the device changes direction.

The torque biofeedback presented to the user may represent only the volitional torque that he/she exerts on the device with the treated limb. However, the load cell or other sensor(s) coupled to the assisted movement device, by virtue of its location in the device, may register not only the user's volitional torque, but also inertial and frictional torques passively arising from the treatment device itself, as well as from the inertia of the limb moved by the device. The sensors coupled to the device may also register any involuntary passive or active torque produced by the user's limb on the device due to spastic contractions of muscles, contractures of the soft tissues in the limb resulting from long-term immobility, or co-contraction of muscles antagonistic to the intended direction of movement, for example. These unintentional, extraneous torques from the treatment device and the users' limbs may be removed from the overall torque biofeedback signal for the users' biofeedback to correspond only to the volitional/intentional assistance they are producing at that moment.

When an assisted movement treatment device moves a joint such that the actively contracting muscle is shortened, it is advantageous for the torque biofeedback to be closely related to the user's level of effort, rather than the user's active torque. By allowing the user to maintain a constant level of effort during joint rotation, rather than the user having to increase effort as the muscle shortens in order to maintain a constant level of torque, the user may be able to perform the assisted movement exercise for longer periods of time without fatiguing.

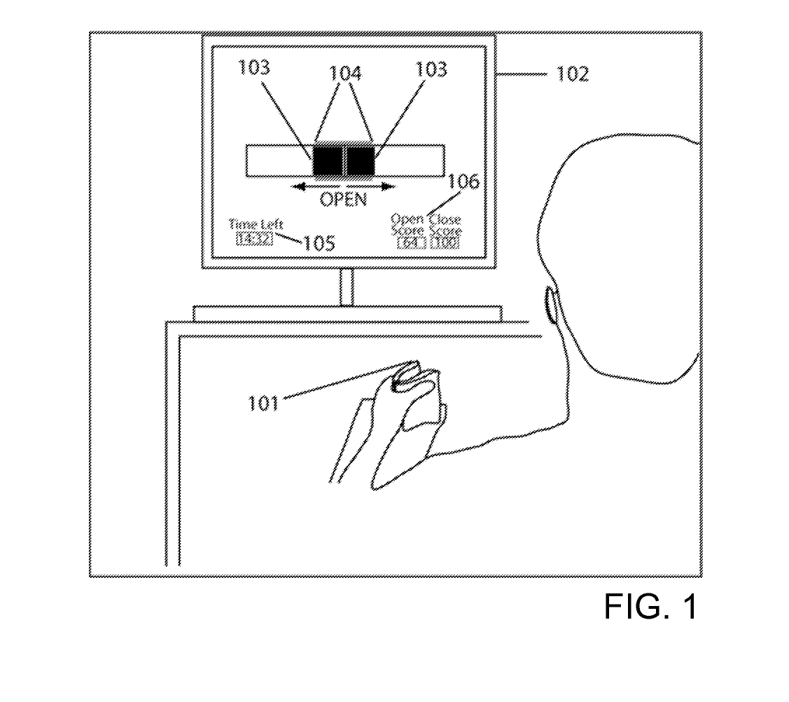

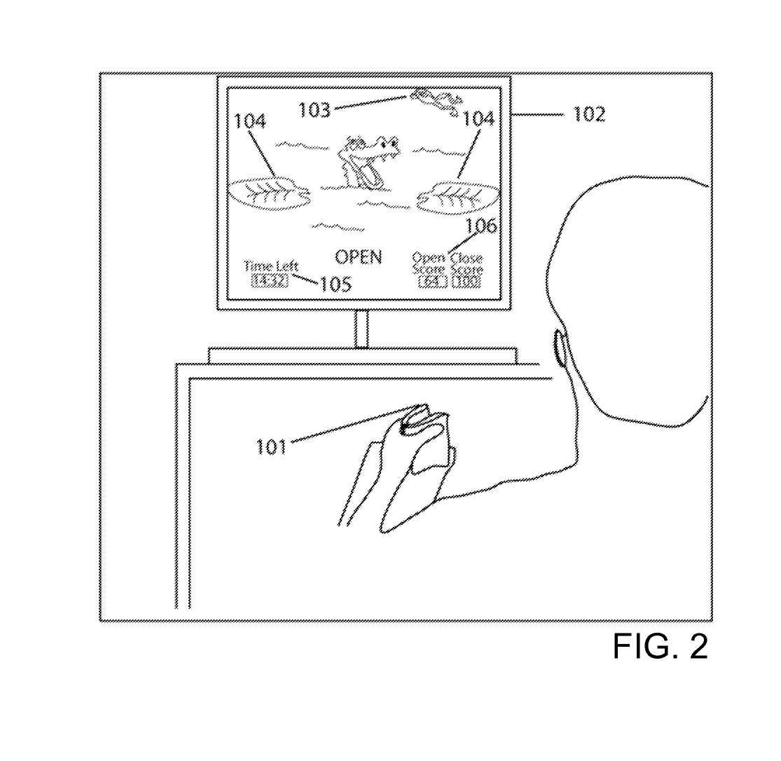

FIGS. 1 and 2 schematically show examples of a user engaged with an assisted movement device and receiving biofeedback in accordance with the disclosure. In particular, FIG. 1 shows a user with the left hand in an assisted movement fixture or device 101, and with visual biofeedback using simple geometrical objects displayed on a computer screen 102. In the example embodiment shown in FIG. 1, biofeedback 103-104 is provided for hand-opening and hand-closing exercise, and the screen graphics depicted correspond to the hand-opening phase of this assisted movement exercise. As the user exerts torque in the hand-opening direction, a filled bar 103 within an open rectangle expands horizontally from a vertical mid-line. Two other narrow bars 104, above and below the open rectangle, show the user the target level of assistive hand torque. The user's task, in this embodiment, is to expand the bar 103 until it exceeds the target level 104, and then to maintain a constant level of effort for the duration of the movement. These applied movements may be slow, e.g., at 5 deg/s, and may last long enough that the user has time to summon up the effort to reach the target, for example, 6 seconds, but limiting the range of the movement, for example, to 30 deg in each direction, in order to not place the user's hand in an uncomfortable position. If the user successfully reaches the target, this success is indicated by an exercise score 106. During an exercise session, this exercise score may indicate the current percentage of assisted movements, in each direction, that the user successfully reached the target level of torque. In some examples, a timer 105 may indicate to the user the time remaining in the exercise session.

FIG. 2 illustrates an example embodiment of torque biofeedback based on a video game format. A video game format may provide substantially the same information to the user as any other biofeedback format, thereby allowing the user to determine whether or not he/she has met the task criteria successfully during each assisted movement. The principal differences between the biofeedback formats depicted in FIGS. 1 and 2 are that, with the video game format, the exercise may be more entertaining, less predictable, and more engaging to the user.

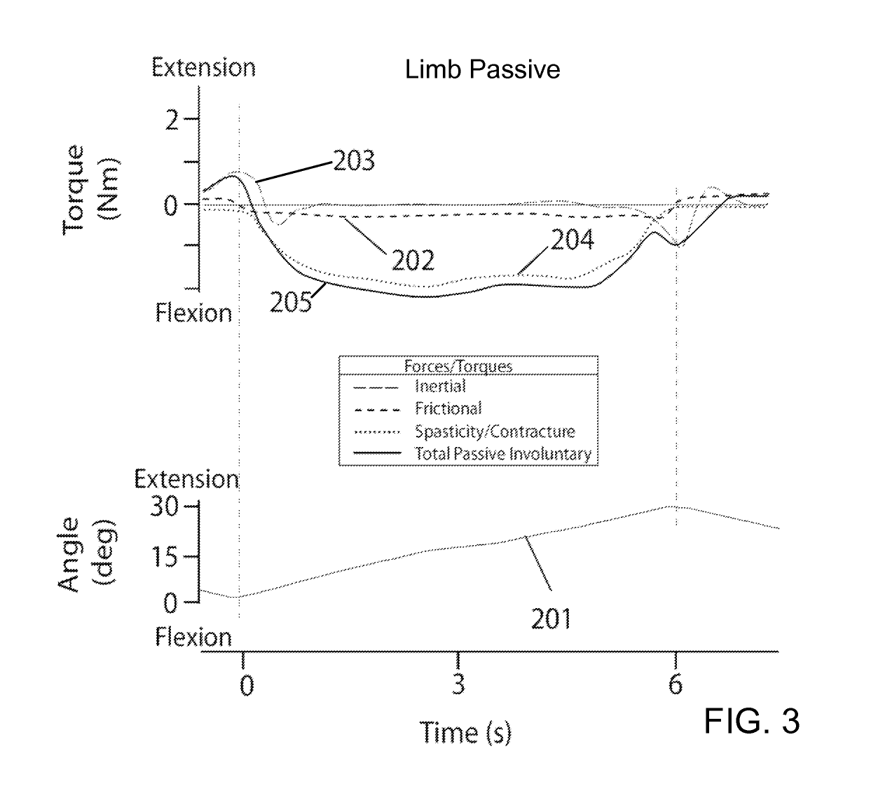

FIG. 3 shows example patterns for passively evoked involuntary torques generated during ½ cycle of a movement applied to a joint or joints of a limb held as relaxed as possible. In particular, FIG. 3 illustrates the involuntary torques generated during a 5-deg/s, 30-deg, 6-second extension movement 201 imposed on a joint by an assisted movement exercise device. In this example, the user keeps the limb as relaxed as possible to ensure that no torque is being produced as a result of the user's volitional effort. The involuntary torques depicted include those due to friction in the assisted movement device 202, inertia of the device and the user's limb in the device 203, and exaggerated tone and contracture in the user's limb 204. The total passively evoked involuntary torque 205 consists of the sum of these 3 sources of torque. The assisted movement exercise device and its software may be capable of performing a passive torque calibration measurement of passively evoked involuntary torque on each user, at the beginning of each user's exercise session.

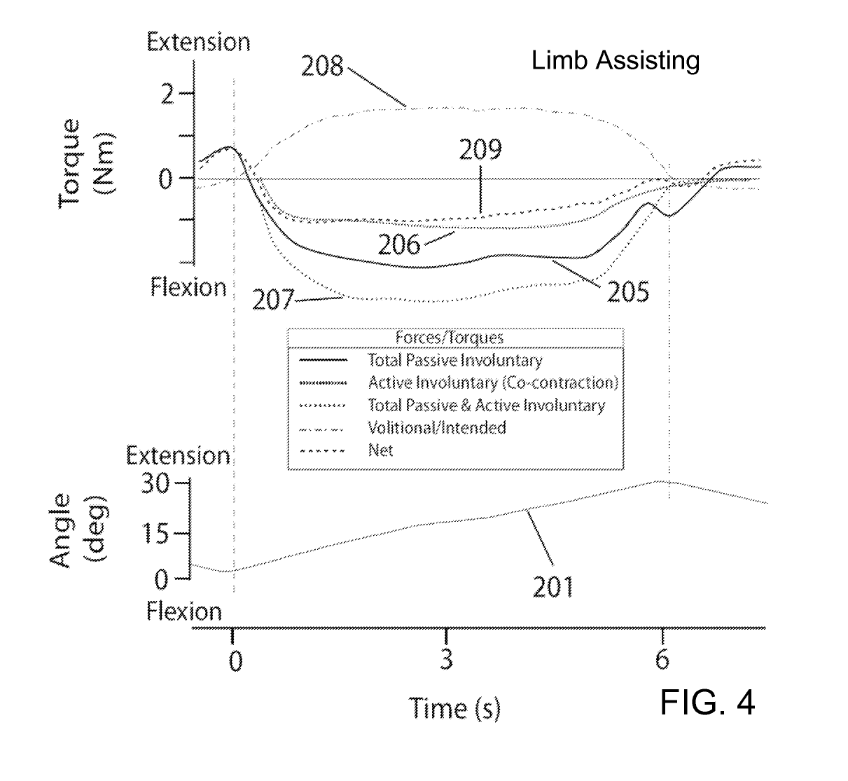

FIG. 4 illustrates a representative pattern of torques produced during assisted movement exercise, while the user is assisting the movement 201. The torques depicted include the total passively evoked involuntary torque 205, as shown in FIG. 3, the volitional/unintentional torque caused by unintentional co-contraction of the antagonist muscle or muscles 206, the sum of all involuntary and unintentional torque including that evoked by passive movement and by co-contraction 207, and the volitional/intended contribution of the user 208. It is this volitional/intentional contribution that preferably is used to drive the biofeedback presented to the user. As shown in FIG. 4, the overall torque produced during this attempted assisted movement in the extension direction is in the flexion instead of the extension direction, because the sum of all involuntary and unintentional oppositional torques exceeds the volitional/intentional torque produced by the user. However, by performing an unintentional torque calibration in order to introduce a torque bias roughly equal and opposite to that caused by unintentional co-contraction of the antagonist muscle 206, and by performing a passive torque calibration that removes the total passive involuntary torque 205, the user will be able to observe biofeedback based solely on the volitional/intentional contraction of the correct muscle, in this example, the extensor.

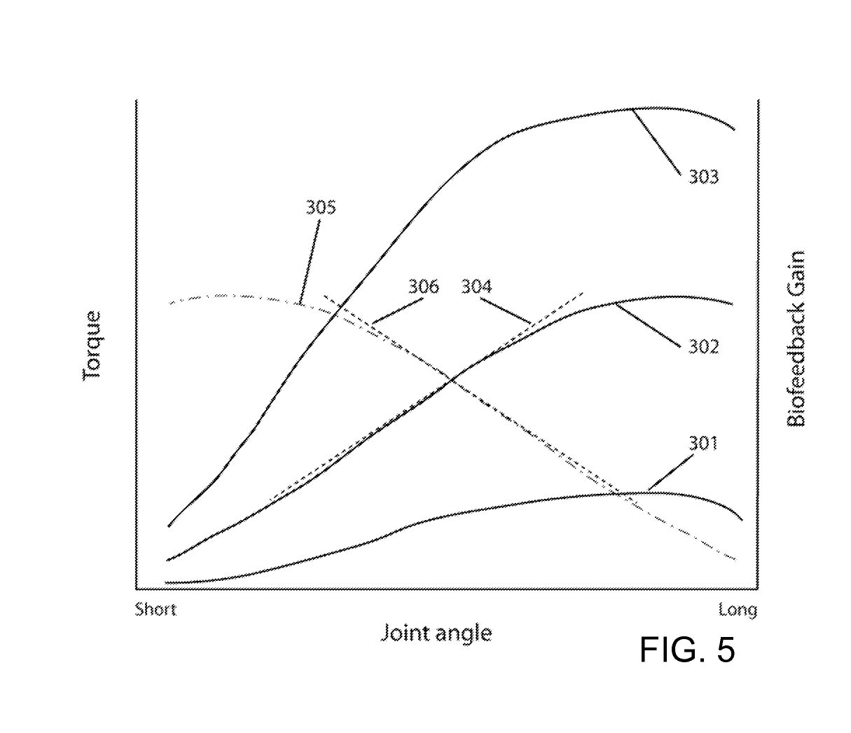

FIG. 5 shows an example graph illustrating the length-tension property of muscle and how it may be used to register the user's effort by changing the gain of the biofeedback. In particular, each of three curves 301-303 shows the length-tension relationship of skeletal muscle at a different level of muscle activation, from weak contractions 301, to moderate contractions 302, to strong contractions 303. Each curve shows that, as muscle length decreases, for example, due to an intentional joint rotation, the active torque generated by the muscle producing the movement also decreases. The relationship between joint torque and muscle length may be quantitatively described by the slope of the relationship, as shown by the dashed line 304, fitted to the intermediate lengths of the muscle.

If simple torque were used to control biofeedback to a user during assisted movement exercise, the shortening of the muscle produced by the movement would decrease the torque produced by the user, requiring increased effort by the user to keep the target level of torque throughout the movement. However, this may lead to muscle fatigue and an inability to complete the exercise.

To compensate for the length-tension property of muscle, and thereby to forestall muscle fatigue during assisted movement exercise, the torque biofeedback may compensate for the length-tension property. For example, the gain of the user's biofeedback 305 may increase correspondingly with the decrease in joint torque produced by muscle shortening resulting in biofeedback related to the user's level of effort. In some examples, this compensation may be performed by a conversion factor, multiplying in software, in real-time, the total volitional/intentional torque, determined according to FIGS. 3 and 4, by the slope of the gain curve 306 shown in FIG. 5. These conversion factors may be selected from any suitable predetermined range or they may be directly measured from a user.

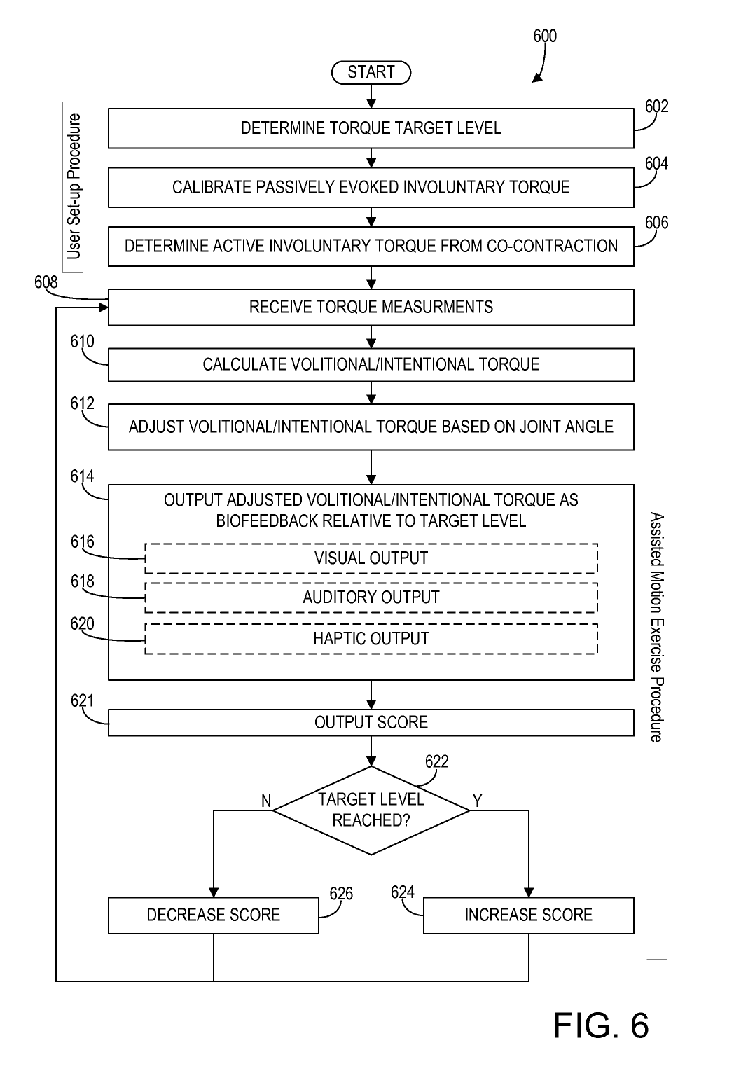

FIG. 6 shows an example method 600 for providing biofeedback during assisted movement exercise while a user is engaged with an assisted movement device, e.g., device 101 shown in FIGS. 1 and 2 described above. Method 600 may be used to extract volitional/intentional components from torque measurements received from an assisted movement device and adjust the volitional/intentional torque to compensate for the length-tension property of muscle in order to present accurate and informative biofeedback to a user during assisted movement exercise.

Method 600 presumes that entry conditions have already been met. Determining if entry conditions are met may include determining if a user is engaged with the assisted movement device, i.e., determining if an assisted movement device is installed around a limb of the user. Thus, determining if entry conditions are met may include receiving signals from one or more sensors coupled to the assisted movement device indicating that the user is engaged with the device. Entry conditions may include any suitable assisted movement exercise initiation conditions. For example, entry conditions may include receiving input from a user to initiate an assisted movement exercise session or a computing system may automatically initiate an assisted movement exercise routine in response to certain conditions such as a completion of a previous assisted movement task.

If entry conditions are met, method 600 proceeds to 602. At 602, method 600 includes determining whether the torque target level needs to be adjusted for the current assisted movement exercise. If the user's strength has not been previously tested, the operator may test strength and enter a baseline target score value, preferably at 25% of maximum in each direction of motion. If the user has previously received assisted movement exercise, in some examples, a user's exercise score from the last assisted movement exercise session may be stored in a storage medium on a computing device. Torque target adjustments are preferentially made so as to maintain an exercise score within a range of 95-100%, but not consistently at 100%, a condition indicating that the exercise is too easy for the user. As another example, the operator may have made a note in the user records at the end of the previous assisted movement exercise that the target level should be adjusted up or down. In any case, prior to assisted movement exercise, the operator may manually adjust the target level during the user set-up procedure per the baseline strength value, previous exercise score, or note in the user records. In another example, the torque target level may be automatically updated by the assisted exercise device, based on the last recorded strength test score, preferably at 25% of maximum in each direction of motion. The torque target value may be adjusted so as to make the exercise challenging but not so difficult that the user becomes too fatigued to complete the exercise session.

A passive torque calibration step may be performed to collect passively evoked involuntary torque measurements from the assisted movement device while the user is passively engaged with the device, i.e., in absence of any volitional torque applied by the user to the device. Thus, at 604, method 600 may include receiving a calibration measurement in the absence of any volitional torque applied by the user to the assisted movement device. In particular, for each position in a plurality of positions of the assisted movement device, a passive torque calibration measurement may be received from the assisted movement device that represents the total passively evoked involuntary torque as shown by 205 in FIG. 3. In one example, the total passively evoked involuntary torque recorded in this calibration at a plurality of positions may then be subtracted from total recorded torque at the same plurality of positions during assisted movement exercise to provide useful biofeedback to the user.

Following the calibration step, method 600 may include 606, in which the exercise device performs an unintentional torque calibration in order to measure the active torque volitionally, but unintentionally produced by a user in the antagonistic muscle or muscles, as described by 209 in FIG. 4. In one example, the operator may place the exercise device in a mode where the joint or joints to be exercised are rendered isometric and joint torque is visualized on the display screen, for example, as shown in FIG. 1. The user may be prompted to produce repetitively small contractions in the direction that causes him/her to co-contract the antagonist muscle while the operator incrementally introduces a voltage bias into the recorded torque signal displayed on the screen. When weak contractions can be observed consistently in the intended direction, the bias voltage level may then be recorded and subsequently subtracted from the torque biofeedback signal during that direction of assisted movement exercise.

During the assisted movement exercise, torque measurements may then be received from the device while the user engages with the device and applies volitional torques to meet target levels during therapy. Thus, for each position in the plurality of positions of the assisted movement exercise device, the following steps may be performed in real-time to automatically provide biofeedback to the user during assisted movement exercise.

In particular, at 608, method 600 includes receiving torque biofeedback from the assisted movement device while the device is in a given position in the plurality of positions of the device. For example, while the user is engaged with the device and is applying a volitional torque to assist in the movement of the device, a torque measurement may be received from one or more sensors coupled to the device.

At 610, method 600 includes calculating a volitional torque from the overall torque measurement. In particular, the passive torque calibration data may be used to remove the passively evoked involuntary torque from the overall torque measurement, e.g., by subtracting passively evoked involuntary torque from the total recorded torque, so that the biofeedback provided to the user indicates only what he/she is doing volitionally. For example, the volitional torque may be determined based on the torque measurement and the calibration measurement at the given position in the plurality of positions of the assisted movement device. As remarked above, determining a volitional torque based on the torque measurement and the calibration measurement at the position may comprise subtracting the calibration measurement from the overall torque measurement to obtain the volitional torque.

At 610, method 600 also includes adjusting the total volitional torque recorded by the torque sensor and adjusted with the passive torque calibration with a second, unintentional torque calibration, to remove volitional, but unintentional, torque produced by contraction of the muscles antagonistic to the current direction of motion during assisted movement exercise. Once both passively evoked involuntary torque and volitional, unintentional torque from antagonist co-contraction have been removed from the overall torque recording, all that remains in the adjusted torque signal is the volitional/intentional torque that the user is generating while assisting the motion of the exercise device.

At 612, method 600 includes adjusting the volitional/intentional torque based on the length of the muscle that the user is attempting to contract in assisting the joint motion to obtain an adjusted volitional/intentional torque. Adjusting the volitional/intentional torque based on a length of a muscle at any of a plurality of joint positions may comprise multiplying the volitional/intentional torque by the gain associated with the length of the muscle of the user at each position to obtain an adjusted volitional/intentional torque. For example, adjusting the volitional/intentional torque based on the length of the user's muscle at the position may comprise increasing the volitional/intentional torque gain in response to a decrease in joint angle to obtain an adjusted volitional/intentional torque value that corresponds more closely to the user's effort than to torque itself. In particular, for a first position in the plurality of positions of the assisted movement device, wherein in the first position the length of the muscle of the user is a first length, the volitional/intentional torque may be adjusted by a first amount; whereas for a second position in the plurality of positions, wherein in the second position the length of the muscle of the user is less than the first length, the volitional/intentional torque gain may be adjusted by a second amount greater than the first amount.

This adjusted volitional/intentional torque relative to a target level may then be presented to users, e.g., visually, aurally, and/or haptically, to inform the users about how well they are complying with the task of assisting the movement. Thus, at 614, method 600 includes outputting the adjusted volitional/intentional torque relative to the target level. For example, at 616, method 600 may include outputting a visual indication of the volitional/intentional torque relative to a visual indication of the target level to a display device. As another example, at 618, method 600 may include outputting an audio indication of the volitional/intentional torque relative to the target level to one or more speakers. For example, one or more sounds may be output to the speakers and/or a frequency or amplitude of one or more sounds may be adjusted to indicate the volitional/intentional torque relative to the target level to the user. As another example, at 620, method 600 may include outputting a haptic indication of the volitional/intentional torque relative to the torque target level. For example, a patch of skin remote from the exercised joint or joints may be stimulated with a mechanical vibrator. The vibration frequency may be increased in proportion to the user's ongoing level of volitional/intentional torque, and a transition from low vibration amplitude, for example, 1 mm peak-to-peak, to a high vibration amplitude, for example, 2-3 mm, may signal the user when his/her effort has caused the target torque to be exceeded.

At 621, method 600 may include outputting a score, e.g., the score 106 shown in FIGS. 1 and 2 described above. For example, a user score may be output to a display device indicating a percentage of assisted movements in which the adjusted volitional torque of the user has reached the target level.

At 622, method 600 includes determining if the target level is reached or exceeded by the user. If the target level is reached, method 600 proceeds to 624 to recalculate the exercise score to obtain an increased score and the resulting higher score is displayed on the display screen. If the target level is not reached, method 600 proceeds to 626 to recalculate the exercise score to obtain a decreased score and the resulting lower score is displayed on the display screen.

Regardless of whether or not the user reached the torque target during the previous assisted movement of the joint, method 600 continues back to 608 to continue receiving torque measurements and extracting and adjusting the torque signal to output to the user during the assisted movement exercise.

In some embodiments, the above described methods and processes may be tied to a computing system including one or more computers. In particular, the methods and processes described herein may be implemented as a computer application, computer service, computer API, computer library, and/or other computer program product.

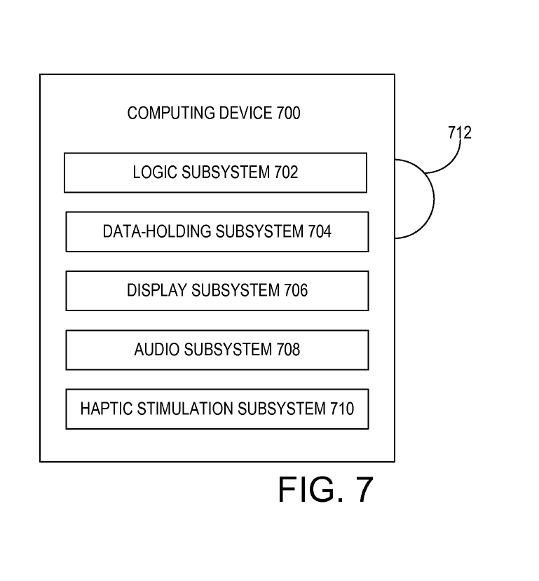

FIG. 7 schematically shows a nonlimiting computing device 700 that may perform one or more of the above described methods and processes. Computing device 700 is shown in simplified form. It is to be understood that virtually any computer architecture may be used without departing from the scope of this disclosure. In different embodiments, computing device 700 may take the form of a mainframe computer, server computer, desktop computer, laptop computer, tablet computer, home entertainment computer, network computing device, mobile computing device, mobile communication device, gaming device, etc.

Computing device 700 includes a logic subsystem 702 and a data-holding subsystem 704. Computing device 700 may optionally include a display subsystem 706, an audio subsystem 708, a haptic stimulation system 710, and/or other components not shown in FIG. 7. Computing device 700 may also optionally include user input devices such as keyboards, mice, game controllers, cameras, microphones, and/or touch screens, for example.

Logic subsystem 702 may include one or more physical devices configured to execute one or more machine-readable instructions. For example, the logic subsystem may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The logic subsystem may include one or more processors that are configured to execute software instructions. Additionally or alternatively, the logic subsystem may include one or more hardware or firmware logic machines configured to execute hardware or firmware instructions. Processors of the logic subsystem may be single core or multicore, and the programs executed thereon may be configured for parallel or distributed processing. The logic subsystem may optionally include individual components that are distributed throughout two or more devices, which may be remotely located and/or configured for coordinated processing. One or more aspects of the logic subsystem may be virtualized and executed by remotely accessible networked computing devices configured in a cloud computing configuration.

Data-holding subsystem 704 may include one or more physical, non-transitory, devices configured to hold data and/or instructions executable by the logic subsystem to implement the herein described methods and processes. When such methods and processes are implemented, the state of data-holding subsystem 704 may be transformed (e.g., to hold different data).

Data-holding subsystem 704 may include removable media and/or built-in devices. Data-holding subsystem 704 may include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 304 may include devices with one or more of the following characteristics: volatile, nonvolatile, dynamic, static, read/write, read-only, random access, sequential access, location addressable, file addressable, and content addressable. In some embodiments, logic subsystem 702 and data-holding subsystem 704 may be integrated into one or more common devices, such as an application specific integrated circuit or a system on a chip.

FIG. 7 also shows an aspect of the data-holding subsystem in the form of removable computer-readable storage media 712, which may be used to store and/or transfer data and/or instructions executable to implement the herein described methods and processes. Removable computer-readable storage media 712 may take the form of CDs, DVDs, HD-DVDs, Blu-Ray Discs, EEPROMs, flash memory cards, and/or floppy disks, among others.

When included, display subsystem 706 may be used to present a visual representation of data held by data-holding subsystem 704. As the herein described methods and processes change the data held by the data-holding subsystem, and thus transform the state of the data-holding subsystem, the state of display subsystem 706 may likewise be transformed to visually represent changes in the underlying data. Display subsystem 706 may include one or more display devices utilizing virtually any type of technology. Such display devices may be combined with logic subsystem 702 and/or data-holding subsystem 704 in a shared enclosure, or such display devices may be peripheral display devices.

When included, audio subsystem 708 may be used to present audio representations of data held by data-holding subsystem 704. As the herein described methods and processes change the data held by the data-holding subsystem, and thus transform the state of the data-holding subsystem, the state of audio subsystem 708 may likewise be transformed to represent changes in the underlying data via audio signals, e.g., via one or more sounds. Audio subsystem 708 may include one or more speakers utilizing virtually any type of technology. Such speaker devices may be combined with logic subsystem 702 and/or data-holding subsystem 704 in a shared enclosure, or such speaker devices may be peripheral speakers.

When included, haptic stimulator subsystem 710 may be used to present haptic representations of data held by data-holding subsystem 704. As the herein described methods and processes change the data held by the data-holding subsystem, and thus transform the state of the data-holding subsystem, the state of haptic subsystem 708 may likewise be transformed to represent changes in the underlying data via haptic signals, e.g., via mechanical vibration of the skin. Haptic subsystem 710 may include one or more vibrators or other skin stimulation mechanisms utilizing virtually any type of technology.

It is to be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated may be performed in the sequence illustrated, in other sequences, in parallel, or in some cases omitted. Likewise, the order of the above-described processes may be changed.

The subject matter of the present disclosure includes all novel and nonobvious combinations and subcombinations of the various processes, systems and configurations, and other features, functions, acts, and/or properties disclosed herein, as well as any and all equivalents thereof.

1. A computerized method for providing real-time biofeedback during assisted movement rehabilitation therapy while a user is engaged with an assisted movement exercise device, the method comprising:
for each position in a plurality of positions of the assisted movement exercise device, receiving a passive torque calibration measurement from the assisted movement device in absence of any volitional torque applied by the user to the assisted movement device; and
for each position in the plurality of positions:
receiving an overall torque measurement from the assisted movement device;
calculating a volitional torque component based on the overall torque measurement and the passive torque calibration measurement at the position;
calculating a volitional/intentional torque component of overall torque based on the overall torque measurement, the volitional torque component, and an unintentional torque calibration measurement of any unintended volitional torque caused by unintended co-contraction of antagonistic muscles at the position;
adjusting the volitional/intentional torque component based on a length of a muscle of the user at the position to obtain an adjusted volitional/intentional torque that represents the user's effort; and
outputting the adjusted volitional/intentional torque component relative to a target level.

2. The method of claim 1, wherein outputting the adjusted volitional/intentional torque component relative to a target level comprises outputting a visual indication of the adjusted volitional/intentional torque component relative to a visual indication of the target level to a display device.

3. The method of claim 1, wherein outputting the adjusted volitional/intentional torque component relative to a target level comprises outputting an audio indication of the adjusted volitional/intentional torque component relative to the target level to one or more speakers.

4. The method of claim 1, wherein outputting the adjusted volitional/intentional torque component relative to a torque target comprises outputting a haptic indication of the adjusted volitional/intentional torque component relative to the target level to one or more skin stimulators.

5. The method of claim 1, wherein calculating the volitional torque component comprises subtracting the passive torque calibration measurement from the overall torque measurement to obtain the volitional torque component.

6. The method of claim 1, wherein calculating the volitional/intentional torque component of overall torque comprises subtracting the unintentional torque calibration measurement from the overall torque measurement to obtain the volitional/intentional torque component of overall torque.

7. The method of claim 1, further comprising correlating an increasing gain with a decreasing length of the muscle of the user based on a desired target level of effort, and wherein adjusting the volitional/intentional torque component based on a length of a muscle of the user at the position comprises multiplying the volitional/intentional torque component by the gain associated with the length of the muscle of the user at the position to obtain an adjusted volitional/intentional torque component.

8. The method of claim 1, further comprising:
for a first position in the plurality of positions, wherein in the first position the length of the muscle of the user is a first length, adjusting the volitional/intentional torque component by a first amount; and
for a second position in the plurality of positions, wherein in the second position the length of the muscle of the user is less than the first length, adjusting the volitional/intentional torque component by a second amount greater than the first amount.

9. The method of claim 1, wherein each position in the plurality of positions of the assisted movement exercise device corresponds to an angle of a joint of the user engaged with the assisted movement exercise device and wherein adjusting the volitional/intentional torque component based on a length of a muscle of the user at the position comprises increasing the volitional/intentional torque component in response to a decrease in joint angle to obtain the adjusted volitional/intentional torque component.

10. The method of claim 1, further comprising outputting a user exercise score indicating a percentage of assisted movements in which the adjusted volitional/intentional torque component of the user reaches the target level, and updating the score in response to the adjusted volitional/intentional torque component reaching the target level.

11. The method of claim 1, further comprising determining a level of strength of the user, and wherein the target level is based on the level of strength of the user.

12. The method of claim 11, wherein the target level is based on the level of strength of the user at a position in the plurality of positions of the device in which the muscle of the user is at a maximum length.

13. The method of claim 11, wherein the target level is further based on an exercise score of the user obtained in a previous assisted movement exercise session.

14. The method of claim 13, further comprising outputting a user exercise score indicating a percentage of assisted movements in which the adjusted volitional/intentional torque component of the user reaches the target level, and updating the score in response to the adjusted volitional/intentional torque component reaching the target level, and, in response to an increase in the level of strength of the user or a leveling off of the exercise score at or around 100%, increasing the target level.

15. The method of claim 1, wherein the calibration measurements and the torque measurements are received from a load cell and/or strain gauge coupled to an axis of rotation of the assisted movement exercise device.

16. A computing system, comprising:
a logic subsystem; and
a data holding subsystem comprising machine-readable instructions stored thereon that are executable by the logic subsystem to:
for each position in a plurality of positions of an assisted movement exercise device engaging a user, receive a passive torque calibration measurement from the device in absence of any volitional torque applied by the user to the assisted movement device; and
for each position in the plurality of positions of the assisted movement exercise device engaging a user, receive an unintentional torque calibration measurement from the device during applied volitional torque by the user to the assisted movement device; and
for each position in the plurality of positions while the user is engaged with the device:
receive a torque measurement from the assisted movement device;
subtract the passive torque calibration measurement at the position from the torque measurement to obtain a volitional torque component;
subtract the unintentional torque calibration measurement at the position from the volitional torque component to obtain a volitional/intentional torque;
multiply the volitional/intentional torque by a gain associated with a length of the muscle of the user at the position to obtain an adjusted volitional/intentional torque representing the user's level of effort; and
output the adjusted volitional/intentional torque relative to a target level.

17. The system of claim 16, further comprising a display device and wherein the data holding subsystem comprising machine-readable instructions stored thereon that are executable by the logic subsystem is further configured to output the adjusted volitional/intentional torque relative to the target level to the display device.

18. The system of claim 16, further comprising one or more speakers and wherein the data holding subsystem comprising machine-readable instructions stored thereon that are executable by the logic subsystem is further configured to output the adjusted volitional/intentional torque relative to the target level to the one or more speakers.

19. The system of claim 16, further comprising one or more haptic stimulators and wherein the data holding subsystem comprising machine-readable instructions stored thereon that are executable by the logic subsystem is further configured to output the adjusted volitional/intentional torque relative to the target level to the one or more haptic stimulators.

20. The system of claim 16, wherein the data holding subsystem comprising machine-readable instructions stored thereon that are executable by the logic subsystem is further configured to receive input indicating a level of strength of the user, and wherein the target level is based on the input indicating the level of strength of the user.

21. The system of claim 16, wherein the passive torque calibration measurements, the unintentional torque calibration measurements, and the torque measurements are received from a load cell and/or strain gauge coupled to an axis of rotation of the assisted movement device.
